(19) United States
(12) Patent Application Publication
(10) Pub. No.: US 2026/0234530 A1
(43) Pub. Date: Aug. 13, 2026

(54) SHAKING DEVICE FOR A CELL CULTURE BOTTLE

(71) Applicant: CellFabs Inc., Tainan City (TW)

(72) Inventors: Yun-Shu Chiou, Tainan City (TW); Shang-Chih Lin, Tainan City (TW)

(73) Assignee: CellFabs Inc., Tainan City (TW)

(21) Appl. No.: 19/049,836

(22) Filed: Feb. 10, 2025

(51) Int. Cl. C12M 3/06; C12M 1/00; C12M 1/34; C12M 1/36; C12M 3/00

(52) U.S. Cl. CPC C12M 27/16; C12M 23/38; C12M 23/48; C12M 41/26; C12M 41/48

(57) ABSTRACT

A shaking device for a cell culture bottle includes: a driver fixed on a base plate, the driver connected to a tilted seat. The bottom center of a shaking platform forms an extension piece, which is positioned on the tilted seat via the extension piece. A limiting frame is movably arranged below the shaking platform, and the limiting frame includes a support structure that can selectively engage with the shaking platform. When the limiting frame rises, the shaking platform is supported horizontally by the support structure, maintaining a level state. Through the downward movement of the limiting frame, the extension piece descends to contact the tilted seat, enabling the driver to move the shaking platform in a three-dimensional circular shaking motion, effectively enhancing the shaking performance.

### BACKGROUND OF THE DISCLOSURE

### 1. Field of the Disclosure

This invention relates to a cell culture device, specifically a shaking device for a cell culture bottle that can achieve three-dimensional orbital shaking movements and effectively enhance shaking uniformity and gas-liquid exchange efficiency in the culture medium.

### 2. Description of the Prior Art

Cell culture plays a critical role in biotechnology applications, encompassing areas such as regenerative medicine, vaccine production, and drug development. Currently, conventional cell culture bottles face significant technical limitations when cultivating suspension cells. These bottles rely on external forces for shaking to promote the flow of the culture medium, primarily to prevent cell aggregation and enhance contact between cells and external air. However, due to the simple structure of these bottles, the shaking typically only produces horizontal liquid flow and fails to create effective vertical disturbance. This results in uneven cell distribution and low culture efficiency.

To address these deficiencies, existing technologies have attempted to improve the internal structure of culture bottles. For instance, designing internal walls with specialized curved surfaces to guide the flow of the culture medium aims to achieve more uniform fluid disturbance. However, such improvements require precise mold design and manufacturing techniques. The complex structures of these bottles can significantly increase production difficulty and, consequently, the cost of production. While these approaches can improve fluid circulation to some extent, their high cost limits practical application, especially in large-scale cell culture operations where expensive bottles cannot entirely replace traditional designs. This necessitates a balanced solution between performance enhancement and cost control. Therefore, this invention proposes a cell culture bottle with innovative structural and functional designs, coupled with a shaking device capable of generating three-dimensional shaking effects. This combination effectively reduces production costs while optimizing shaking performance.

In light of these challenges, the inventor, drawing upon years of experience in the manufacturing, development, and design of related products, meticulously designed and assessed solutions to the aforementioned objectives, finally achieving a truly viable invention.

### SUMMARY OF THE DISCLOSURE

The technical problem this invention aims to address is to provide a shaking device for a cell culture bottle that resolves the deficiencies present in the prior art.

A driver is fixed to a plate body and connected to an inclined seat. A rocking plate has an extension piece formed at the center of its bottom and is mounted on the inclined seat via the extension piece. A limiting frame is movably positioned below the rocking plate and can selectively support the rocking plate through a support body. When the limiting frame is lowered, the rocking plate is inclined and does not contact the support body, allowing the inclined seat and the extension piece to interact, causing the rocking plate to rotate in an inclined circular motion. When the limiting frame is raised, the rocking plate is supported in a horizontal state by the support body.

One end of the extension piece is fixed to the rocking plate, while the other end is recessed with a conical hole. The inclined seat has a protruding cylinder, which is arranged non-coaxially with the driver. The cylinder passes through the conical hole to restrict the movement of the extension piece.

The extension piece is integrally formed on the bottom surface of the rocking plate and is recessed with a conical hole. The inclined seat has a protruding cylinder, which is arranged non-coaxially with the driver, and the cylinder extends through the conical hole.

Multiple springs are connected between the driver and the extension piece. The tensile force of the springs pulls the extension piece toward the inclined seat.

The inclined seat is formed with two parallel side plates, with an inclined surface between them. One of the side plates has an oval hole. One end of the extension piece is connected to the rocking plate through a shaft rod and at least one bearing, while the other end is movably positioned between the two side plates. The extension piece is fitted with the oval hole via a rotating shaft, allowing the rocking plate to swing around the rotating shaft at the oval hole.

The plate body is fixed with at least one telescopic element, which drives a slanted block. The slanted surface of the slanted block pushes the limiting frame, creating upward and downward height changes. Additionally, the plate body is equipped with multiple touch switches. When the slanted block contacts a touch switch, it stops the drive in a unidirectional manner.

The invention further includes a frame for securing multiple plate bodies. The frame has sliding grooves on both sides corresponding to the plate body, allowing the plate body to slide along the grooves. Both the frame and the plate body are provided with at least one perforation, through which a pin is inserted to secure the plate body to the frame.

The invention further includes a cell culture bottle, which is placed on the rocking plate. The cell culture bottle has multiple positioning recesses recessed at its bottom, while the rocking plate is correspondingly equipped with multiple positioning protrusions. The combination of the positioning recesses and positioning protrusions prevents the cell culture bottle from detaching from the rocking plate.

The cell culture bottle has multiple cylinders protruding inward from its bottom. Each cylinder is fixed with a guide vane, and the guide vanes extend upward from the cylinders. Multiple guide vanes are arranged in a spiral configuration along the inner wall of the cell culture bottle. Additionally, the cell culture bottle is sealed with a cap, which is attached with a breathable waterproof membrane. The breathable waterproof membrane allows air to flow into the cell culture bottle while maintaining a sealed environment.

A pH sensor is fixed to the exterior of the cap. The pH sensor includes a sensing rod, which is inserted into the interior of the cell culture bottle.

The cell culture bottle features a coupling member protruding at the center of its bottom. This coupling member is fitted with a flow guide blade, which directs the culture medium from the center toward the direction of the guide vanes. The coupling member is composed of multiple elastic pillars, while the flow guide blade has a ring at its center, which is mounted on and secured to the elastic pillars.

The first main objective of this invention is to keep the rocking plate in a horizontal position, supported by the support body, when the limiting frame is raised. In this state, the inclined seat and the extension piece remain uncontacted. This enables easy placement and retrieval of the cell culture bottle from the rocking plate. When the limiting frame is lowered, the extension piece moves downward to contact the inclined seat, and the driver actuates the extension piece via the inclined seat. This causes the rocking plate to perform a three-dimensional circular rocking motion, effectively enhancing the shaking performance.

The second main objective of this invention is to use the spiral arrangement of all guide vanes in the cell culture bottle to create a vortex flow structure. During shaking, the culture medium flows outward under centrifugal force, is guided upward along the guide vanes, and then recirculates back to the center of the cell culture bottle from the top of the culture medium. This ensures fluid disturbance in the vertical direction, prevents cell clumping, and enhances the gas-liquid exchange efficiency of the culture medium, thereby promoting cell growth.

Other objectives, advantages, and novel features of the present invention will become more apparent from the following detailed description and accompanying drawings.

### BRIEF DESCRIPTION OF THE DRAWINGS

The present invention will be best understood by referring to the following detailed description of one illustrative embodiment in conjunction with the accompanying drawings, in which

FIG. 1 is a perspective view of the present invention;

FIG. 2 is an exploded perspective view of the present invention;

FIG. 3 is a sectional view showing the state of the limiting frame in a raised position according to the present invention;

FIG. 4 is a schematic view of the rocking motion of the present invention in the state where the limiting frame is lowered;

FIG. 5 is a schematic view showing the present invention installed in a frame;

FIG. 6 is a perspective view of the cell culture bottle according to the present invention;

FIG. 7 is an exploded perspective view of the cell culture bottle according to the present invention;

FIG. 8 is a schematic view showing the cell culture bottle in a rocking state according to the present invention;

FIG. 9 is a schematic view of the present invention with a spring installed;

FIG. 10 is a perspective view of another embodiment of the present invention;

FIG. 11 is an exploded perspective view of another embodiment of the present invention;

FIG. 12 is a schematic view of the rocking motion in the state where the limiting frame is lowered according to another embodiment of the present invention;

FIG. 13 is a schematic view showing the state of the limiting frame in a raised position according to another embodiment of the present invention;

FIG. 14 is a perspective view of yet another embodiment of the present invention;

FIG. 15 is an exploded perspective view of yet another embodiment of the present invention;

FIG. 16 is a schematic view showing the state where the limiting frame is lowered according to yet another embodiment of the present invention; and

FIG. 17 is a schematic view showing the state of the limiting frame in a raised position according to yet another embodiment of the present invention.

### DETAILED DESCRIPTION

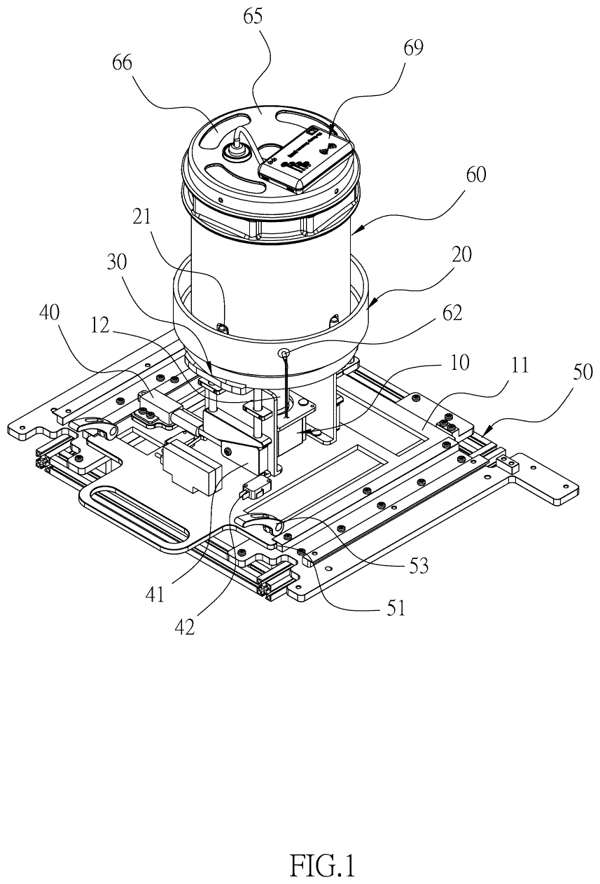

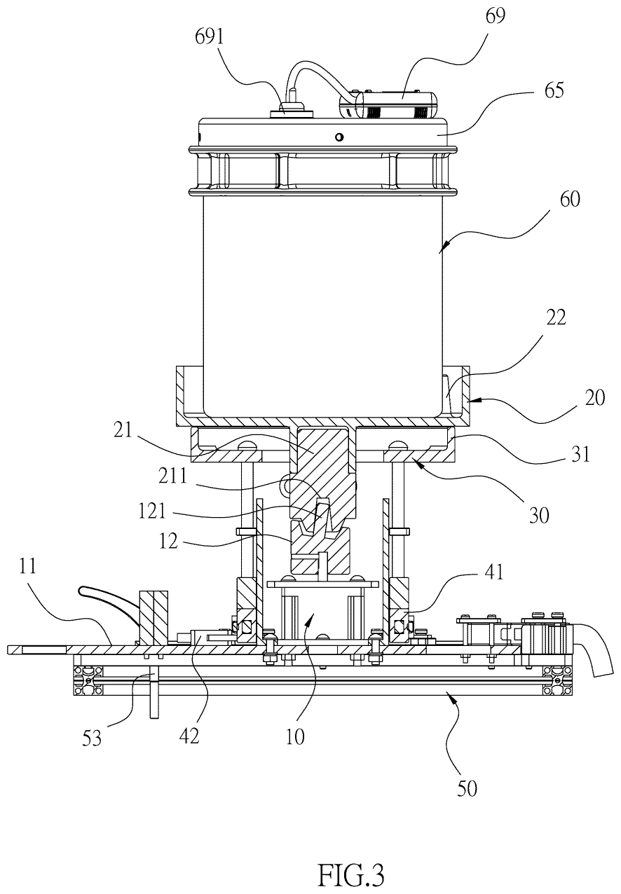

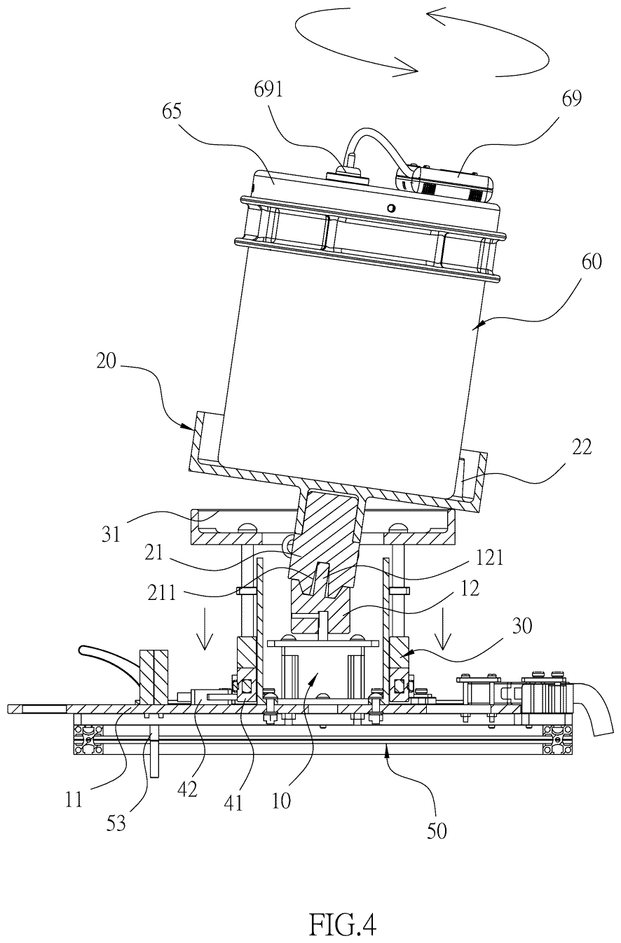

Referring to FIGS. 1, 2, and 3, a shaking device for a cell culture bottle includes a driver 10, a rocking plate 20, and a limiting frame 30. The driver 10 is fixed to a plate body 11 and connected to an inclined seat 12. The driver 10 can be a stepper motor that drives the inclined seat 12 to rotate. The center bottom of the rocking plate 20 forms an extension piece 21, which is positioned at the inclined seat 12. The extension piece 21 is fixed at one end to the rocking plate 20, and the other end has a recessed conical hole 211. The inclined seat 12 is equipped with a protruding cylinder 121, which is non-coaxially arranged with the driver 10. The cylinder 121 passes through the conical hole 211, thereby restricting the movement of the extension piece 21. The driver 10 drives the cylinder 121 to rotate in a circular motion around a conical axis, and the cylinder 121 passing through the conical hole 211 limits the extension piece 21, causing the rocking plate 20 to rotate in an inclined circular motion. A limiting frame 30 is movably installed below the rocking plate 20. The limiting frame 30 includes a support body 31, which can selectively engage with the rocking plate 20. When the limiting frame 30 descends, the rocking plate 20 tilts and does not contact the support body 31. The inclined seat 12 and the extension piece 21 interact with each other. Referring to FIG. 4, when the limiting frame 30 ascends, the rocking plate 20 is supported in a horizontal position by the support body 31, and the inclined seat 12 and the extension piece 21 do not interact. Through the ascending motion of the limiting frame 30, the rocking plate 20 remains horizontal, allowing objects placed on the rocking plate 20 to be easily handled. Subsequently, the descending motion of the limiting frame 30 allows the extension piece 21 to contact the inclined seat 12. The driver 10 uses the inclined seat 12 to drive the extension piece 21, causing the rocking plate 20 to perform a three-dimensional circular rocking motion, thereby effectively improving the shaking performance.

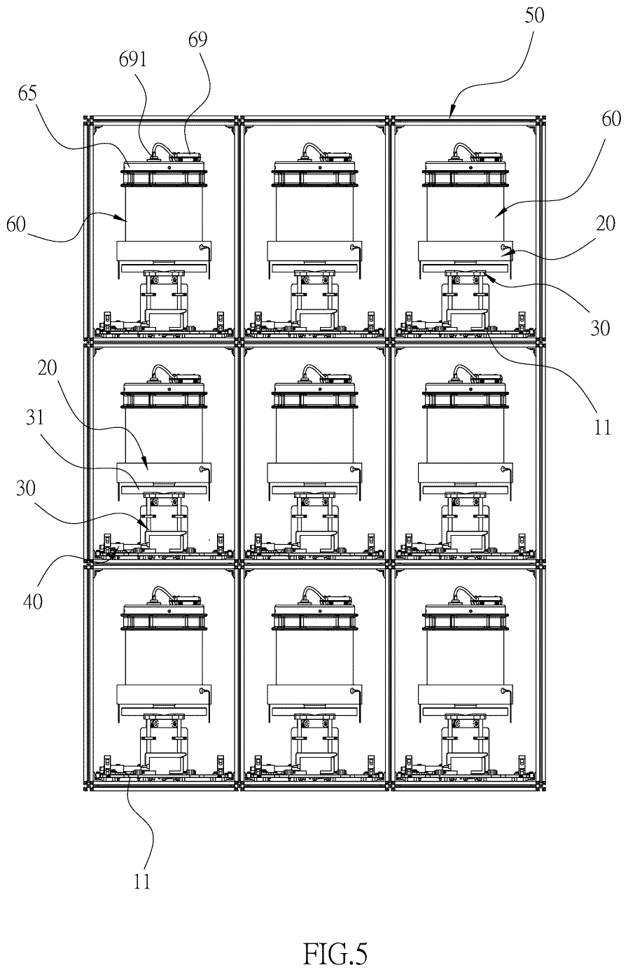

Referring further to FIG. 1 through 4, the plate body 11 is equipped with at least one telescopic element 40, which may be a pneumatic cylinder, hydraulic cylinder, or electric cylinder. The telescopic element 40 drives a slanted block 41, which is linearly displaced by the telescopic element 40. The slanted surface of the slanted block 41 pushes the limiting frame 30 to vary its height, enabling upward and downward motion. The plate body 11 is also equipped with multiple touch switches 42. When the slanted block 41 contacts the touch switch 42, it triggers a unidirectional stop mechanism. Referring to FIG. 5, the device further includes a frame 50 for securing multiple plate bodies 11. The frame 50 has sliding grooves 51 on both sides, allowing the plate body 11 to slide into position within the grooves 51. Both the frame 50 and the plate body 11 feature at least one perforation 111, 52. A pin 53 passes through these perforations to secure the plate body 11 to the frame 50. In summary, the frame 50 can be configured to form a cell culture chamber capable of holding numerous shaking devices. By automating the control of shaking frequency, duration, and speed, the system facilitates the development of large-scale automated cell culture factories. The core technology lies in the coordinated use of the inclined seat 12, extension piece 21, and limiting frame 30, enabling a simplified and cost-effective structure to achieve enhanced shaking performance.

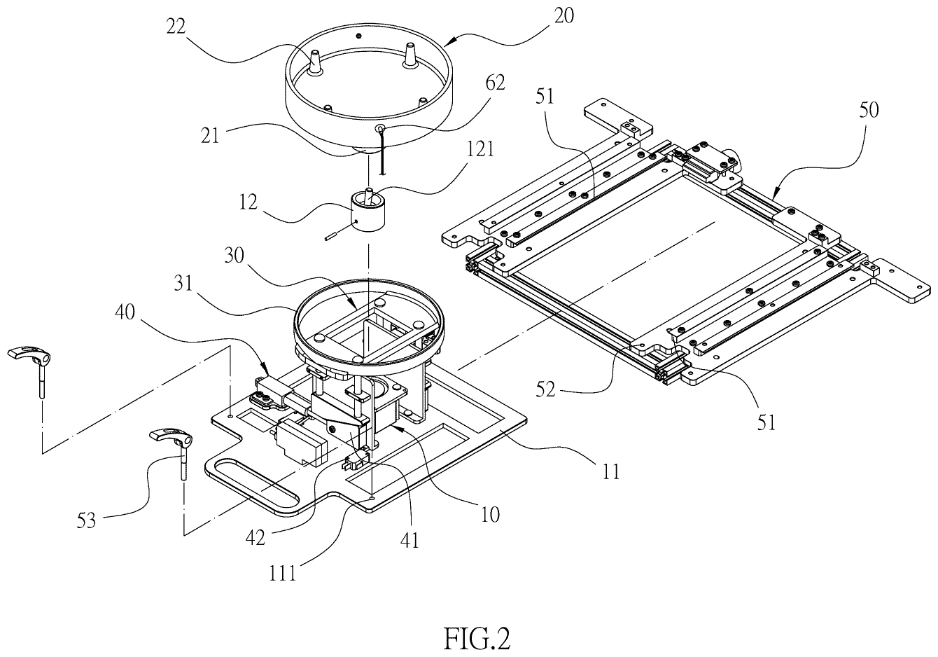

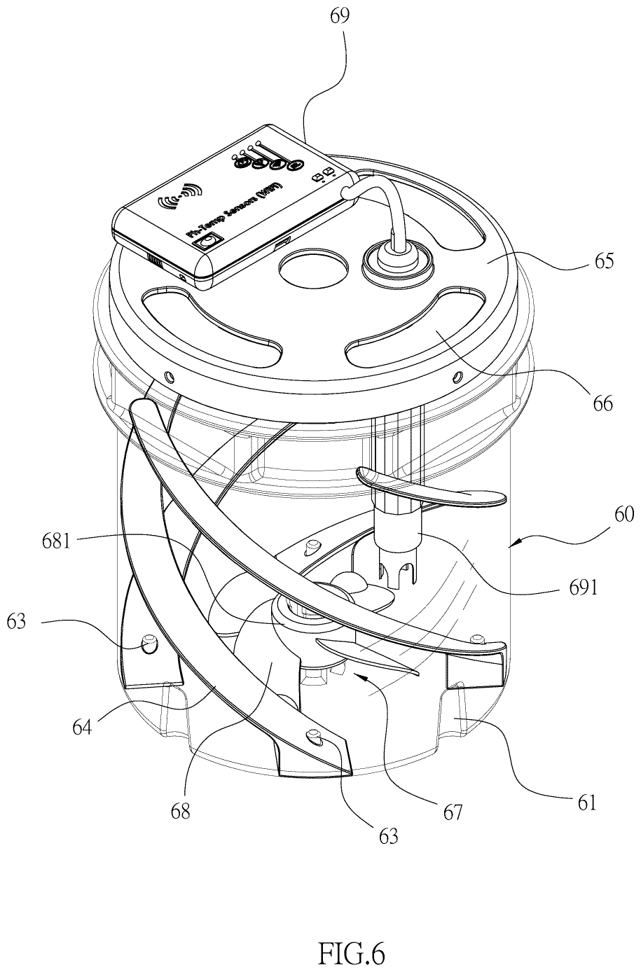

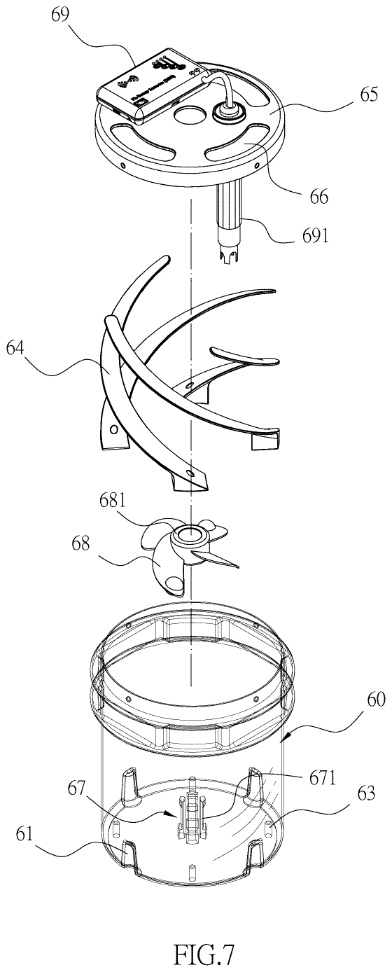

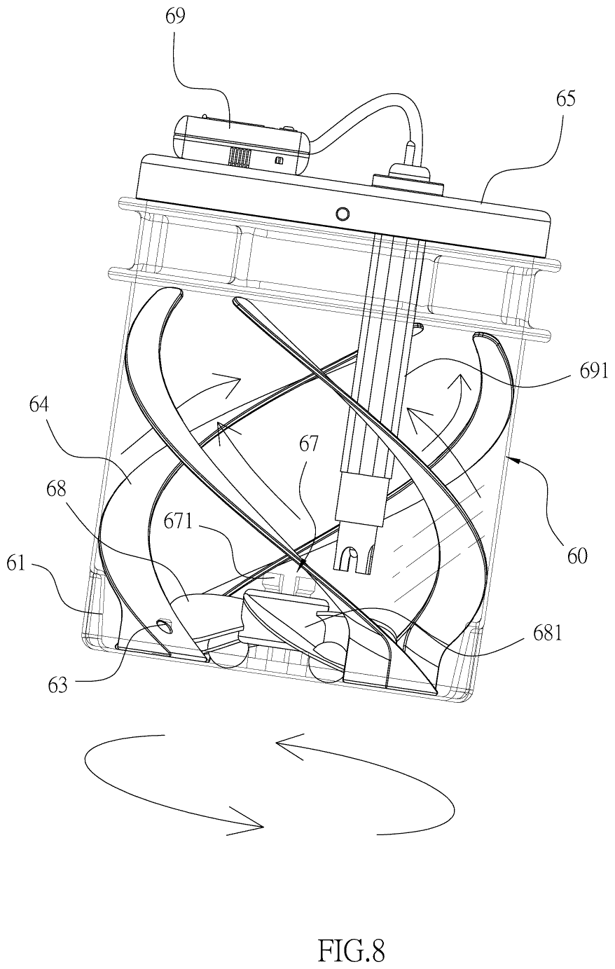

Referring to FIGS. 6, 7, 8, and FIG. 2, the present invention further includes a cell culture bottle 60 positioned on the rocking plate 20. The bottom of the cell culture bottle 60 is recessed with multiple positioning recesses 61, and the rocking plate 20 is correspondingly equipped with multiple positioning protrusions 22. The engagement of the positioning recesses 61 and the positioning protrusions 22 prevents the cell culture bottle 60 from dislodging from the rocking plate 20. Additionally, the device includes an infrared sensor 62 that projects onto the body of the cell culture bottle 60 to determine whether the bottle is placed on the rocking plate 20. The infrared sensor 62, fixed to the rocking plate 20, can also immediately detect if the cell culture bottle 60 becomes dislodged. Further details include the bottom of the cell culture bottle 60 being inwardly convex with multiple cylinders 63. Each cylinder 63 is fixed with a guide vane 64 that extends upward from the cylinder 63. Multiple guide vanes 64 are helically arranged along the inner wall of the cell culture bottle 60, forming a vortex flow structure. During rocking, the culture liquid flows outward under centrifugal force, guided upward along the guide vanes 64, and recirculates to the center of the cell culture bottle 60 from above the liquid. This design facilitates vertical fluid perturbation, preventing cell clumping and enhancing gas-liquid exchange efficiency in the culture liquid, thereby promoting cell growth. Additionally, the center bottom of the cell culture bottle 60 has a convex coupling member 67, which is fitted with a flow guide blade 68. The flow guide blade 68 directs the culture liquid from the center toward the guide vanes 64. The coupling member 67 consists of multiple elastic pillars 671, with the center of the flow guide blade 68 forming a ring 681. The ring 681 is positioned to fit over and be limited by the elastic pillars 671, preventing the flow guide blade 68 from dislodging while allowing it to be rotated by the flowing culture liquid. This cooperative structure of the flow guide blade 68 and the coupling member 67 forms a fluid-guiding assembly, enabling rapid assembly of the flow guide blade 68 and allowing it to rotate freely. This configuration effectively stirs cells deposited at the bottom of the cell culture bottle 60, redistributing them into suspension within the culture liquid and enhancing the fluid dynamics of the culture liquid.

The cell culture bottle 60 is sealed with a cap 65, which is fitted with a breathable waterproof membrane 66. This breathable waterproof membrane 66 allows air to flow freely into the interior of the cell culture bottle 60 while blocking moisture penetration. This feature ensures gas exchange within the culture environment while preventing liquid leakage and bacterial contamination, thereby enhancing the stability of the culture process. The cap 65 is externally equipped with a pH sensor 69, which includes a sensing rod 691 inserted into the interior of the cell culture bottle 60. The sensing rod 691 is designed for real-time monitoring of the pH level of the culture liquid, enabling users to track pH changes without opening the cell culture bottle 60. This allows adjustments to the shaking duration or liquid replacement operations based on the collected data. The pH measurement results can also be transmitted via wired or wireless communication for further utilization.

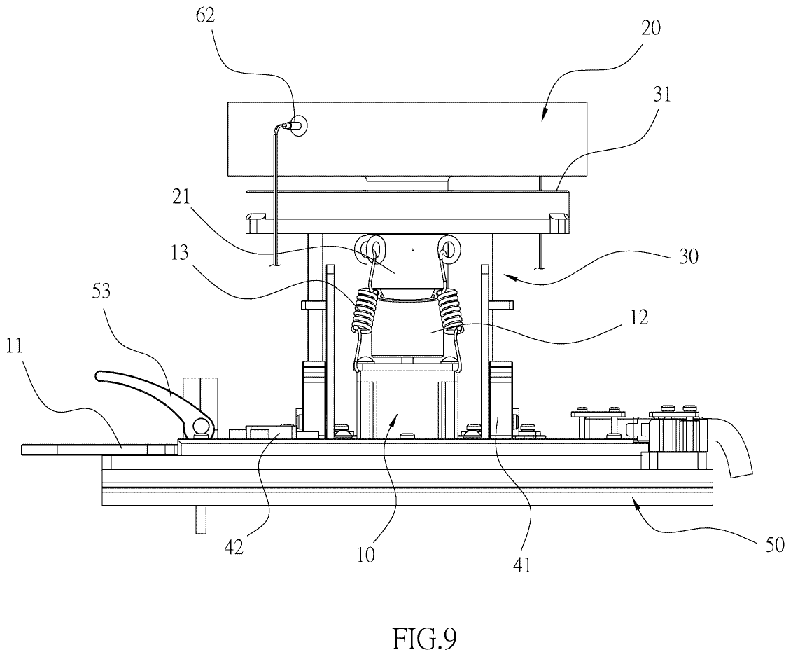

Referring to FIG. 9, multiple springs 13 are connected between the driver 10 and the extension piece 21. The tension of the springs 13 pulls the extension piece 21 toward the inclined seat 12. When the limiting frame 30 is raised to elevate the rocking plate 20, the springs 13 stretch and press the rocking plate 20 tightly against the support body 31 of the limiting frame 30. This configuration effectively prevents lateral displacement of the rocking plate 20. When the limiting frame 30 is lowered, the rocking plate 20 presses firmly against the inclined seat 12. This prevents unstable rocking or bouncing of the rocking plate 20, thereby improving the stability of the rocking motion during operation.

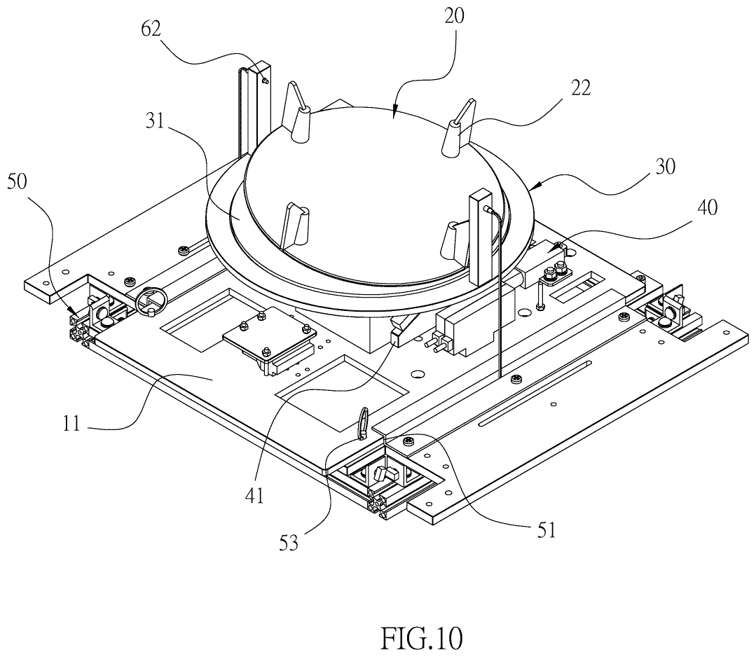

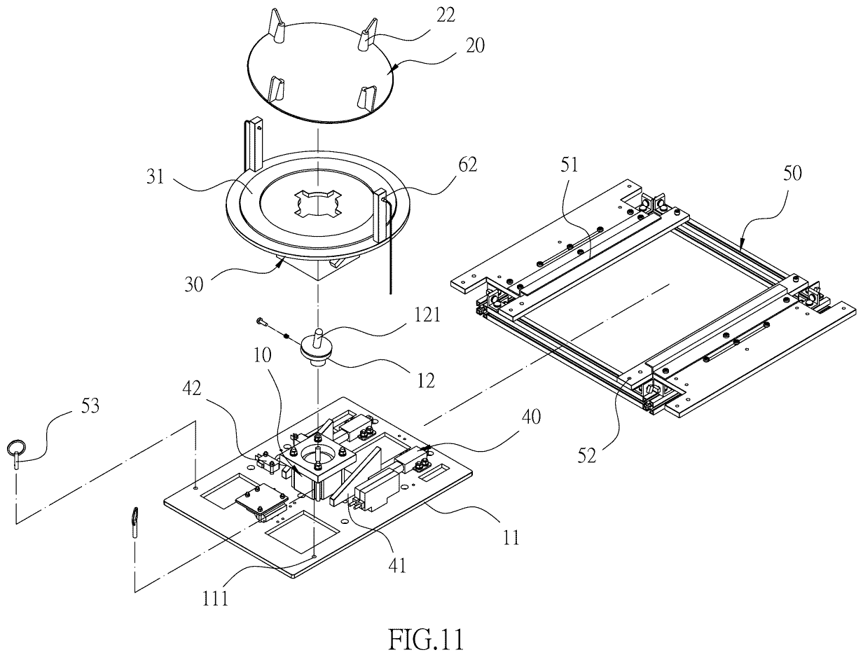

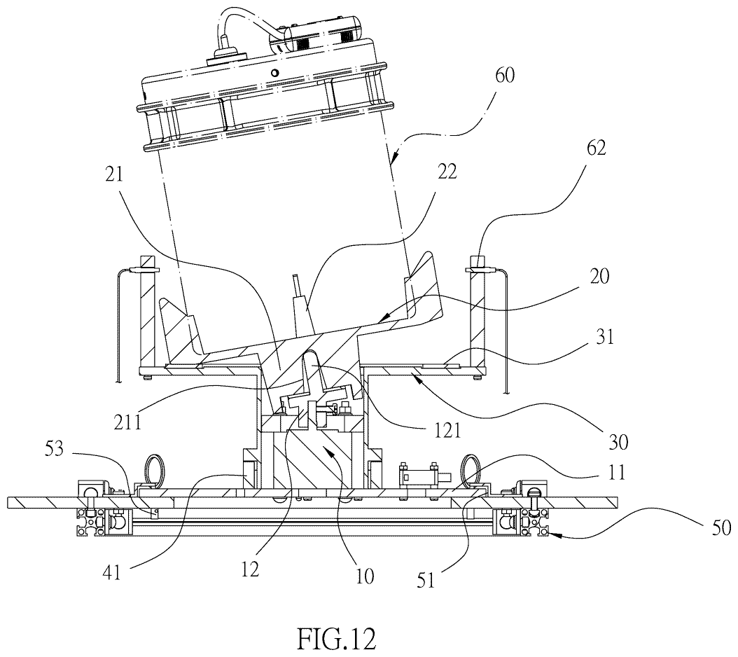

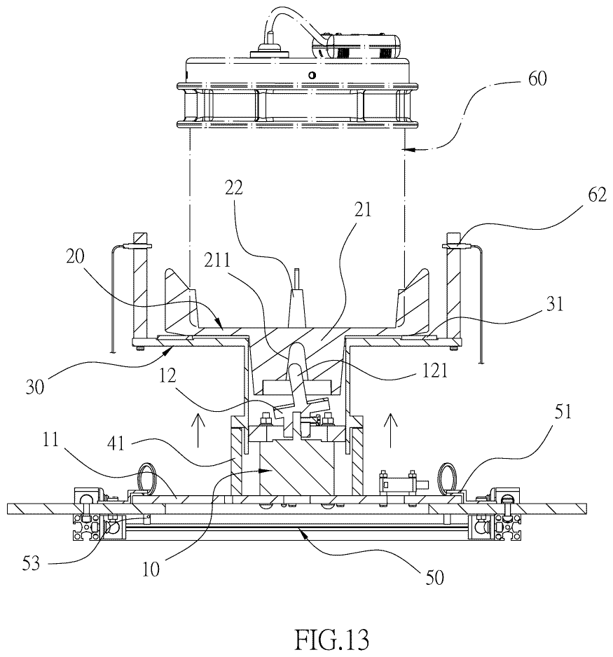

In another embodiment of the present invention, as shown in FIG. 10 through 13, a shaking device for a cell culture bottle includes a driver 10, a rocking plate 20, and a limiting frame 30. The driver 10 is fixed to a plate body 11, which can be slidably installed within the frame structure of a frame 50. The driver 10 is connected to an inclined seat 12. The bottom center of the rocking plate 20 is formed with an extension piece 21, which is integrally molded on the underside of the rocking plate 20. The extension piece 21 has a recessed conical hole 211. The inclined seat 12 has a protruding cylinder 121, which is arranged eccentrically with respect to the axis of the driver 10. The cylinder 121 is inserted into the conical hole 211. When the driver 10 drives the rotation of the cylinder 121, it performs a conical circling motion around the axis. The limiting frame 30 is movably installed beneath the rocking plate 20. The limiting frame 30 includes a support body 31 that can optionally contact the rocking plate 20. When the limiting frame 30 is lowered, the rocking plate 20 is inclined and does not contact the support body 31. This configuration effectively reduces the overall height of the assembly, allowing the frame 50 to accommodate more shaking devices within the same volume, thereby significantly improving space utilization. An infrared sensor 62 is fixed to the limiting frame 30. The infrared sensor 62 emits a beam directed at the body of the cell culture bottle 60 to determine whether the cell culture bottle 60 is placed on the rocking plate 20. To summarize, when the limiting frame 30 is raised, the rocking plate 20 is maintained in a horizontal position supported by the support body 31, and the inclined seat 12 does not contact the extension piece 21. This raised position of the limiting frame 30 facilitates easy placement or removal of the cell culture bottle 60 on the rocking plate 20. When the limiting frame 30 is lowered, the extension piece 21 descends to contact the inclined seat 12. The driver 10, through the inclined seat 12, drives the extension piece 21 to create a three-dimensional circling rocking motion on the rocking plate 20, thereby effectively enhancing the shaking performance.

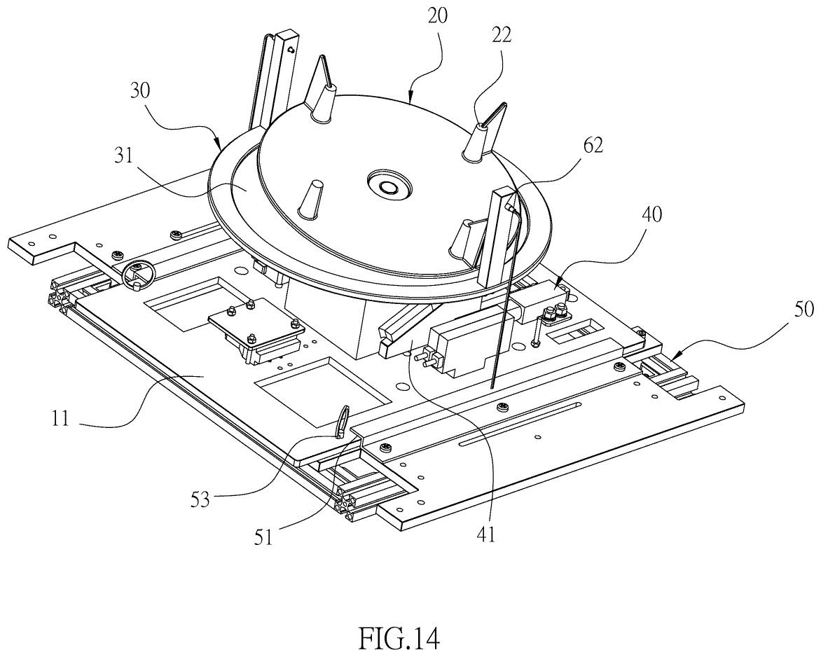

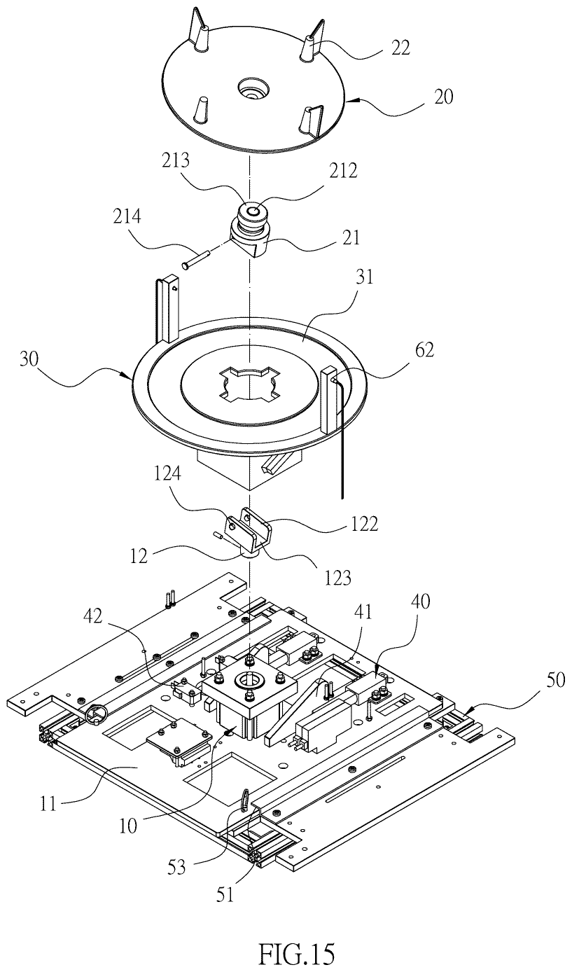

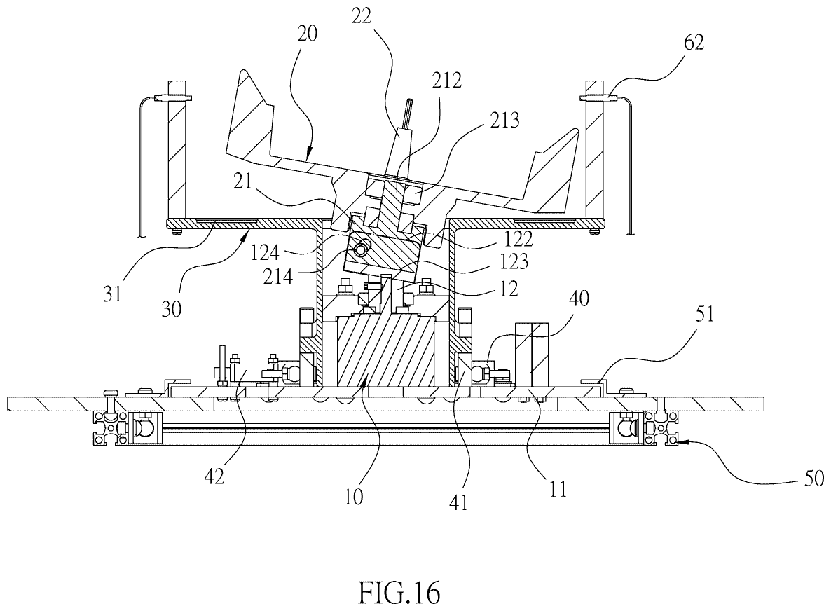

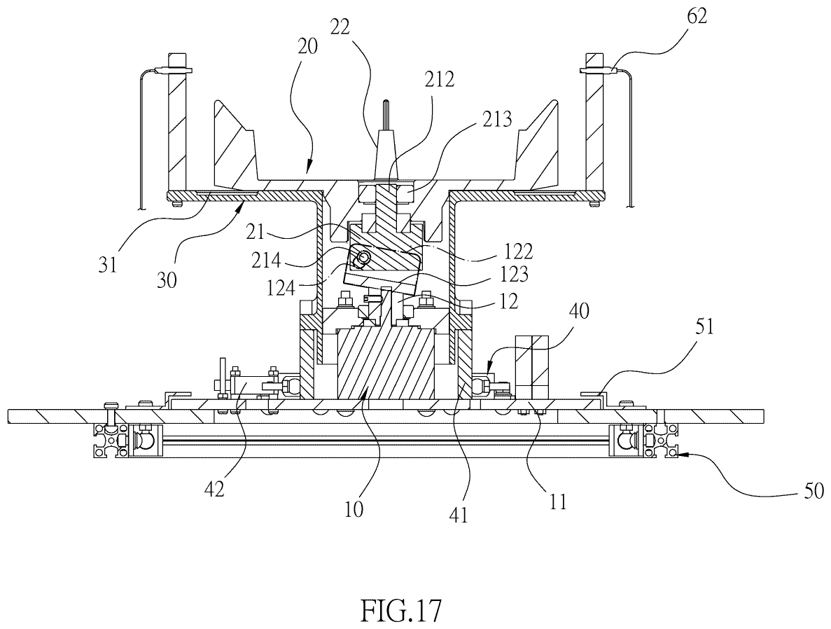

In yet another embodiment of the present invention, as shown in FIG. 14 through 17, a shaking device for a cell culture bottle includes a driver 10, a rocking plate 20, and a limiting frame 30. The main difference lies in the connection relationship between the driver 10 and the rocking plate 20. The inclined seat 12 of the driver 10 is parallelly formed with two side plates 122, and an inclined surface 123 is formed between the two side plates 122. One side of the side plates 122 has an oval hole 124. One end of the extension piece 21 of the rocking plate 20 is connected to the rocking plate 20 via a shaft rod 212 and at least one bearing 213. The other end of the extension piece 21 is movably placed between the two side plates 122. The extension piece 21 is fitted with the oval hole 124 and has a rotating shaft 214 passing through it, allowing the rocking plate 20 to swing using the rotating shaft 214 and the oval hole 124 as pivot points. When the limiting frame 30 is raised, the rocking plate 20 is maintained in a horizontal position supported by the support body 31. At this point, the extension piece 21 is lifted and oscillates along the oval hole 124 via the rotating shaft 214, causing the extension piece 21 to separate from the inclined surface 123. This allows the rocking plate 20 to remain in a horizontal state through the upward movement of the limiting frame 30. When the limiting frame 30 is lowered, the extension piece 21 swings downward and contacts the inclined surface 123. At this time, the rocking plate 20 tilts, and the driver 10, through the inclined seat 12, drives the extension piece 21, causing the rocking plate 20 to perform a three-dimensional circling rocking motion. This configuration effectively enhances the shaking performance.

It should be realized that the above description is only some preferred embodiments of the present invention and should not be deemed as limitations of implementing the present invention. All substantially equivalent variations and modifications which employ the concepts disclosed in this specification and the appended claims should fall within the scope of the present invention.

What is claimed is:

1. A shaking device for a cell culture bottle, comprising;
a driver fixed to a plate body and connected to an inclined seat;
a rocking plate with an extension piece formed at its central bottom, the rocking plate being positioned on the inclined seat via the extension piece; and
a limiting frame movably positioned beneath the rocking plate, the limiting frame selectively contacts the rocking plate through a support body, when the limiting frame descends, the rocking plate tilts without contacting the support body, and the inclined seat interacts with the extension piece to cause the rocking plate to perform a tilted circular motion, when the limiting frame ascends, the rocking plate is supported in a horizontal position by the support body.

2. The shaking device for cell culture bottle of claim 1, wherein one end of the extension piece is fixed to the rocking plate, and the other end of the extension piece has a conical hole; the inclined seat is formed with a protruding cylinder, which is eccentrically aligned with the driver, the cylinder passes through the conical hole to restrict the movement of the extension piece.

3. The shaking device for cell culture bottle of claim 2, wherein a plurality of springs connects the driver and the extension piece, the tension of the springs pulls the extension piece toward the inclined seat.

4. The shaking device for cell culture bottle of claim 1, wherein the extension piece is integrally formed on the bottom surface of the rocking plate, and the extension piece has a conical hole; the inclined seat is formed with a protruding cylinder, which is eccentrically aligned with the driver, the cylinder passes through the conical hole.

5. The shaking device for cell culture bottle of claim 4, wherein a plurality of springs connects the driver and the extension piece, the tension of the springs pulls the extension piece toward the inclined seat.

6. The shaking device for cell culture bottle of claim 1, wherein the inclined seat is formed with two parallel side plates, and an inclined surface is formed between the two side plates; one side of the side plates is provided with an oval hole, one end of the extension piece is connected to the rocking plate with a shaft rod and at least one bearing, and the other end of the extension piece is movably arranged between the two side plates, the extension piece is provided with a rotating shaft through the oval hole, allowing the rocking plate to swing using the rotating shaft and the oval hole.

7. The shaking device for cell culture bottle of claim 1, wherein the plate body is fixed with at least one telescopic element, the telescopic element is connected to drive a slanted block, and the slanted surface of the slanted block pushes the limiting frame to create height changes for upward and downward motion; the plate body is also fixed with a plurality of touch switches, which form a unidirectional stop drive when the slanted block contacts the touch switches.

8. The shaking device for cell culture bottle of claim 1, further comprising a frame, wherein the frame is used to fix multiple plate bodies; the frame is provided with sliding grooves on both sides corresponding to the plate body, allowing both sides of the plate body to slide in the sliding grooves, the frame and the plate body are each provided with at least one perforation, and a pin passes through the perforations to fix the plate body to the frame.

9. The shaking device for cell culture bottle of claim 1, further comprising a cell culture bottle, wherein the cell culture bottle is placed on the rocking plate; the bottom of the cell culture bottle is recessed with multiple positioning recesses, and the rocking plate is correspondingly provided with multiple positioning protrusions, the combination of the positioning recesses and the positioning protrusions prevents the cell culture bottle from detaching from the rocking plate.

10. The shaking device for cell culture bottle of claim 9, further comprising an infrared sensor, wherein the infrared sensor projects onto the body of the cell culture bottle to determine whether the rocking plate is holding the cell culture bottle, and the infrared sensor can be fixed to either the limiting frame or the rocking plate.

11. The shaking device for cell culture bottle of claim 9, wherein the bottom of the cell culture bottle is inwardly protruded with multiple cylinders, each cylinder is fixed with a guide vane, the guide vane extends upward from the cylinder, multiple guide vanes are arranged spirally along the inner wall surface of the cell culture bottle; the cell culture bottle is capped with a cap, the cap is externally fixed with a pH sensor, the pH sensor includes a sensing rod, and the sensing rod is inserted into the cell culture bottle.
